(19) United States
(12) Patent Application Publication
(10) Pub. No.: US 2004/0265249 A1
(43) Pub. Date: Dec. 30, 2004

(54) Composition for protecting skin from damaging effects of ultraviolet light

(75) Inventor: James Demetrios G. Arquette, Tempe, AZ (US)

Correspondence Address: Dale F. Regelman, Law Office of Dale F. Regelman, P.C., 4231 S. Fremont Avenue, Tucson, AZ, 85714, US

(73) Assignee: Desert Whale Jojoba Company

(21) Appl. No.: 10/602,188

(22) Filed: Jun. 24, 2003

(51) Int. Cl. A61K 7/42; A61K 35/78

(52) U.S. Cl. 424/59; 424/757

(57) ABSTRACT

A topical composition for the protection of skin from damage caused by ultraviolet radiation. The composition includes a jojoba extract that absorbs ultraviolet radiation having one or more wavelengths between about 290 nanometers and about 400 nanometers. The composition further includes a jojoba extract comprising a plurality of antioxidant compounds comprising a ferulic acid moiety. The topical compositions are incorporated into sunscreen products, soap, moisturizing lotions, skin toners, and other skin care products.

### FIELD OF THE INVENTION

This invention relates to a topical antioxidant and UV absorbing composition for the protection and treatment of human skin, particularly skin that is exposed to harmful ultraviolet radiation, and a method for preparing that composition.

### BACKGROUND OF THE INVENTION

Darker skin pigmentation is considered desirable by many persons, socially and aesthetically. At present, the most common method for darkening the skin is through suntanning, using either natural sunlight or specially designed ultraviolet (UV) light sources, e.g., tanning lamps. It is known, however, that extended exposure of human skin to ultraviolet light has adverse consequences, both in the short term and in the long term. Specifically, in the short term, individuals exposed to UV risk a painful sunburn and keratitis. In the long term, extended exposure to ultraviolet radiation can result in photoaging and “leathery” skin, and can further result in various forms of skin cancer and ultimately death.

Fair-skinned individuals are particularly susceptible to sun-induced skin disorders and cancers. For example, they face a higher risk of melanoma (skin cancer), and often incur photo-aging or dermatoheliosis, a condition characterized by wrinkling, irregular pigmentation, and surface roughness. However, even darker skinned individuals exposed to prolonged sunlight incur a high risk of skin cancer and exacerbated aging.

Nevertheless, the continued desirability of the suntan look, has resulted in a wide range of UV protection sunscreen agents. Such sunscreen agents are typically suspended in a cream, lotion, gel, mousses, waxed based sticks, aerosols, and alcohol sticks for topical application to the skin. Numerous companies market a large assortment of popular sunscreen lotions with varying degrees of sun block which extend the body's normal resistance to UV radiation.

However, sunscreen products are not perfect in their mode of action. There is no single sunscreen agent that is capable of absorbing all of the harmful wavelengths striking the skin. Higher Sun Protection Factor (SPF) formulations address this problem by including a combination of sunscreen agents in the formulation. Even when using a combination of sunscreen agents, however, these products do not provide complete protection, particularly from the longer ultraviolet wavelengths. Although these longer wavelengths do not readily elicit many of the acute damaging effects commonly attributed to ultraviolet light exposure, recent research indicates that these wavelengths can create free radicals in the skin. These free radicals may be responsible for the premature aging of the skin commonly linked to ultraviolet light exposure.

According to the free radical theory of premature aging of the skin, ultraviolet light can produce reactive oxygen species (“ROS”) that damage the skin. ROS are a collection of reactive free radicals produced from the oxygen molecule, and include singlet oxygen, the superoxide radical, hydrogen peroxide, and the hydroxyl radical, as well as the reaction products produced by these free radicals. Due to their reactivity, ROS relatively indiscriminately react with other molecules, and generate a cascade of harmful free radical reactions in the skin.

The skin possesses defense mechanisms against the generation of ROS. These defenses include the presence of enzymes such as superoxide dismutase, catalase, glutathione transferase, glutathione peroxidase and glutathione reductase, as well as antioxidants such as tocopherols; ubiquinone, ubiquinol, ascorbic acid and dehydroascorbic acid. Unfortunately, ultraviolet light entering the skin can easily overwhelm these defense systems, such that the amount of superoxide dismutase and glutathione transferase in the skin declines significantly upon irradiation with solar simulated ultraviolet light. Simultaneous with the loss of these reducing enzymes, there is a dramatic increase in conjugated double bonds formed in the skin from the linoleates present in cell membranes. There is also an increase in thiobarbituric acid reactive substances present in the skin, which represent a collection of molecules that are formed from ROS.

Sunburn cells are prematurely dead keratinocytes that are produced in skin as a result of ultraviolet light exposure. The nexus between ROS and the formation of sunburn cells remains unknown. However, given the fact that ROS produce negative effects upon molecules in the cell membranes as well as in proteins including enzymes that-control most cellular activity, it has been suggested that ROS could play a potentially important role in the formation of sunburn cells. Even with advances in recent years in the protection of skin from harmful ultraviolet radiation, the epidemic of skin cancer and skin damage from the effects of this radiation has continued unabated. The loss of portions of the ozone layer from environmental pollution is believed to have contributed to an increase in ambient ultraviolet radiation that reaches exposed skin. Many skin protection preparations that could prevent sun damage have an unacceptable odor or texture that discourages their more frequent use, and many of the available skin protectants do not sufficiently protect the skin from these many mechanisms of injury. Hence there is a significant public health need for commercially acceptable or improved preparations that can be topically applied to human and animal skin, to offset the harmful effects of ultraviolet radiation.

Applicants' topical composition comprises a plurality of naturally-occurring materials derived from the jojoba plant. Applicant's jojoba extract absorbs ultraviolet radiation having wavelengths between about 290 nanometers and about 400 nanometers, and comprises a plurality of naturally-occurring antioxidant compounds.

### SUMMARY OF THE INVENTION

Applicants' invention includes a topical composition for reducing skin damage induced by ultraviolet radiation comprising a jojoba extract, where that jojoba extract absorbs ultraviolet radiation at a plurality of wavelengths between about 290 nanometers and about 400 nanometers. In certain embodiments, Applicant's topical composition further includes one or more antioxidant compounds.

Applicant's invention further includes a method to form Applicant's topical composition. Applicant's method first provides jojoba plant parts and removes up to about 90 weight percent of the jojoba oil disposed in those jojoba plant parts. Those jojoba plant parts are then milled to a powder and extracted with one or more polar solvents to form an extract which absorbs ultraviolet radiation at a plurality of wavelengths between about 290 nanometers and about 400 nanometers, and which further includes one or more antioxidant compounds. In certain embodiments, one or more additional skin protectants that reduce the skin damage caused by ultraviolet light are added to Applicant's jojoba extract.

### BRIEF DESCRIPTION OF THE DRAWINGS

The invention will be better understood from a reading of the following detailed description taken in conjunction with the drawings in which like reference designators are used to designate like elements, and in which:

FIG. 1 is a flow chart summarizing the steps of Applicant's method to prepare his Applicant's topical composition;

FIG. 2 is a transmittance plot showing the UV absorption of a first embodiment of Applicant's jojoba extract;

FIG. 3 is an absorbance plot showing the UV absorption of the first embodiment of Applicant's jojoba extract; and

FIG. 4 is a transmittance plot showing the UV absorption of a second embodiment of Applicant's jojoba extract.

### DETAILED DESCRIPTION OF THE PREFERRED EMBODIMENTS

This invention is described in preferred embodiments in the following description with reference to the Figures, in which like numbers represent the same or similar elements.

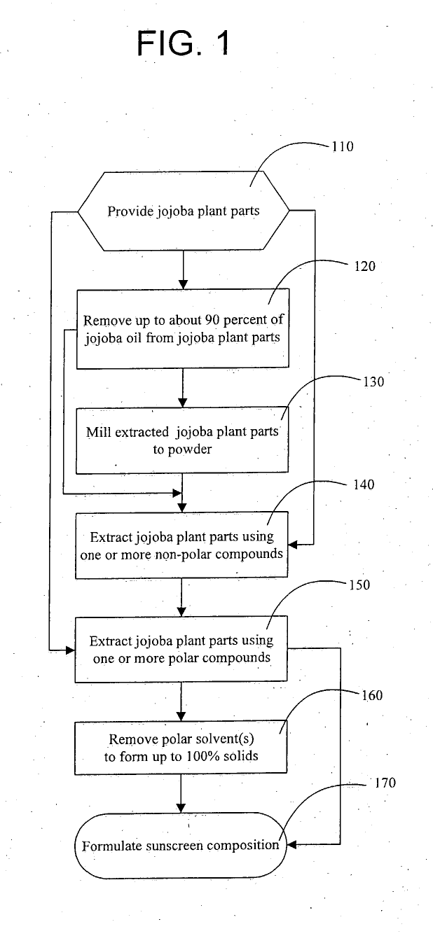

FIG. 1 summarizes Applicant's method to prepare his sunscreen composition comprising a jojoba extract. By “jojoba extract,” Applicant means one or more materials extracted from one or more jojoba plant parts, where those one or more extracted materials are substantially free of jojoba oil.

By “jojoba oil,” Applicant means a mixture of naturally-occurring straight chain esters obtained from the jojoba plant. For example, jojoba seed contains about 50 weight percent of a yellow oil commonly referred to as jojoba oil. In contrast to other vegetable oils which comprise a mixture of triglycerides, jojoba oil comprises a mixture of long-chain esters.

Referring now to FIG. 1, in step 110 Applicant's method provides one or more parts of the jojoba plant. Such jojoba plant parts include, without limitation, seed, pressed seed, hulls, bark, roots, leaves, stems, and the like, but not jojoba oil alone. In certain embodiments, step 110 includes providing jojoba seed. In certain embodiments, jojoba seed is provided by the Desert Whale Jojoba Company of Tucson, Ariz. Jojoba seed comprises approximately 50 percent jojoba oil. The various parts of the jojoba plant, including jojoba seed, further comprise a complex mixture of jojoba oil, jojoba proteins, carbohydrates, Simmondsin, Simmondsin derivatives, and other phytochemicals. In certain embodiments, Applicant's method transitions from step 110 to step 120. In certain embodiments, Applicants' method transitions from step 110 to step 140. In certain embodiments, Applicant's method transitions from step 110 to step 150.

In step 120, Applicant's method removes up to about 90 percent of the jojoba oil disposed in the plant parts of step 110. In certain embodiments, step 120 comprises mechanically pressing the jojoba plant parts of step 110. In certain embodiments, such mechanical pressing is performed using an expeller apparatus. The solid material remaining after removal of jojoba oil from jojoba plant parts is sometimes referred to as “jojoba meal.” This jojoba meal comprises up to about 12 percent residual jojoba oil in addition to a complex mixture of jojoba proteins, sugars, Simmondsin, and other phytochemicals. In certain embodiments; Applicant's method transitions from step 120 to step 130. In certain embodiments, Applicant's method transitions from step 120 to step 140.

As noted above, the various parts of the jojoba plant include a variety of jojoba proteins and amino acids. Table I recites the amino-acid composition of the jojoba meal of step 120.

TABLE I

| AMINO ACID | WEIGHT PERCENT |
|---|---|
| Lysine | 1.45 |
| Histidine | 0.61 |
| Arginine | 1.95 |
| Aspartic Acid | 2.82 |
| Threonine | 1.41 |
| Serine | 1.53 |
| Glutamic Acid | 3.36 |
| Proline | 1.44 |
| Glycine | 2.45 |
| Alanine | 1.19 |
| Valine | 1.54 |
| Methionine | 0.35 |
| Isoleucine | 1.03 |
| Leucine | 2.02 |
| Tyrosine | 1.07 |
| Phenylalanine | 1.23 |
| Cystine | 0.8 |
| Tryptophan | 0.32 |
| TOTAL | 26.57 |

In step 130, the jojoba plant parts of step 120 are milled to a powder. In certain embodiments, the milled jojoba plant parts of step 130 have an average particle size of about 50 microns with a standard deviation of 1.83. In certain embodiments, the milled jojoba plant parts of step 130 have no particles larger than about 180 microns.

In step 140, the jojoba plant parts of step 120 are extracted using one or more non-polar compounds. By a non-polar compound, Applicant means a material having a dielectric constant of about 2 or less. Such non-polar compounds include, without limitation, pentane, hexane, cyclohexane, and the like.

In certain embodiments, step 140 further includes using super critical CO₂ to extract the jojoba meal. Super critical carbon dioxide comprises highly pressurized carbon dioxide. At pressures of 250 to 350 times atmospheric pressure, CO₂ takes on the density of a liquid and the viscosity of a gas, making it an efficient solvent. In its pressurized state, CO₂ is pumped into a sealed chamber containing jojoba meal, where it is allowed to circulate to remove the residual jojoba oil. Two of the major advantages of CO₂ are that it does not leave a chemical residue and it has a minimal to no effect on the structure of the extracted jojoba oil. In certain embodiments, step 140 includes extracting the pressed jojoba plant parts with one or more solvents having a dielectric constant of about 6 or less. Such solvents include, without limitation, methyl formate, methyl acetate, ethyl acetate, ethers, and halogenated alkyls.

In step 150, the one or more jojoba plant parts of either step 110 or step 140 are extracted with one or more polar compounds. By “polar compound,” Applicant means a material having a dielectric constant of about 10 or greater. In certain embodiments, step 150 includes extracting the one or more plant parts with water. In certain embodiments, step 150 includes extracting the one or more jojoba plant part with water in combination with one or more alcohols and/or one or more diols and/or one or more polyols.

Examples I and II are presented to further illustrate to persons skilled in the art how to make and use the invention and to identify certain embodiments thereof These examples are not intended as limitations, however, upon the scope of the invention, which is defined only by the appended claims.

### EXAMPLE I

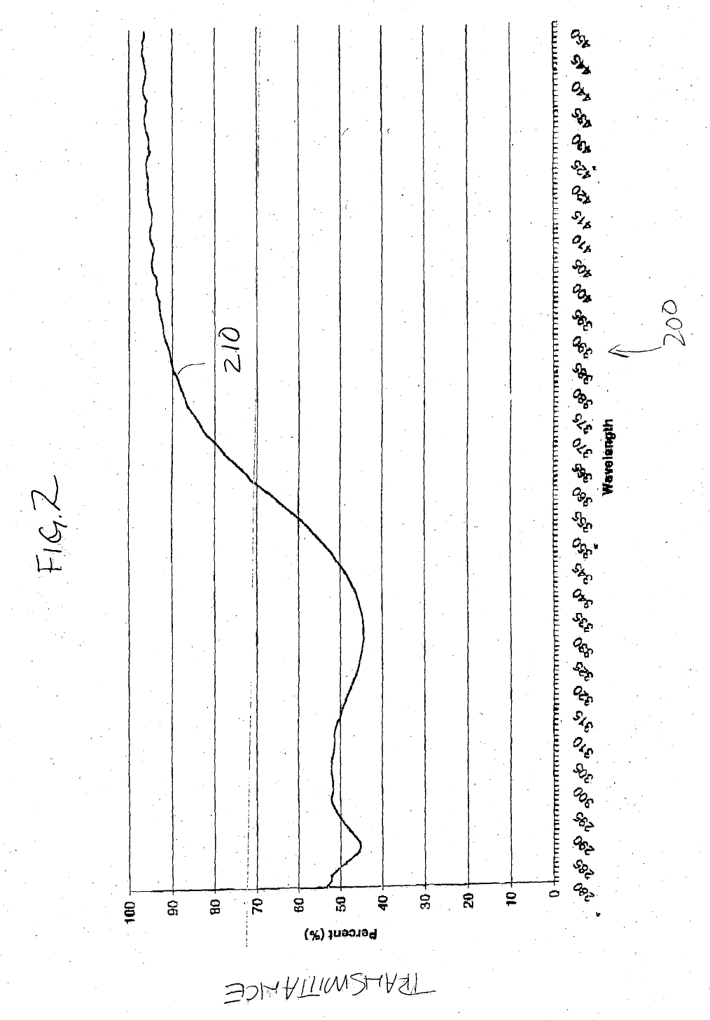

Referring now to FIG. 2, graph 200 shows the transmittance plot 210 for a jojoba extract formed by extracting jojoba meal with 80 weight percent ethanol/20 weight percent water solution. As curve 210 shows, the ethanolic water extract of this embodiment of step 150 absorbs ultraviolet radiation at a plurality of wavelengths between 290 nanometers and 400 nanometers.

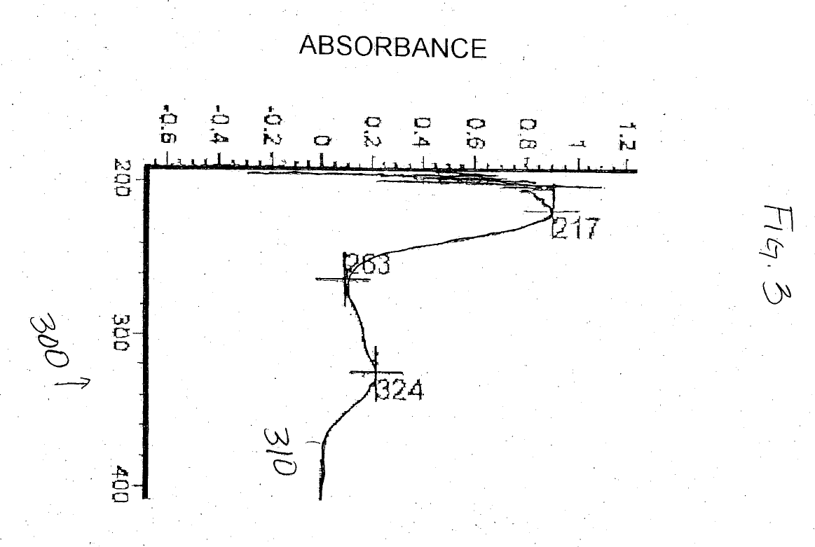

Referring now to FIG. 3, graph 300 shows an absorbance plot for a jojoba extract formed using an 80/20 ethanol/water solution. As curve 310 shows, the ethanolic water extract of this embodiment of step 150 absorbs ultraviolet radiation at a plurality of wavelengths between 290 nanometers and 380 nanometers with a maximum absorption in that range at about 324 nanometers.

### EXAMPLE II

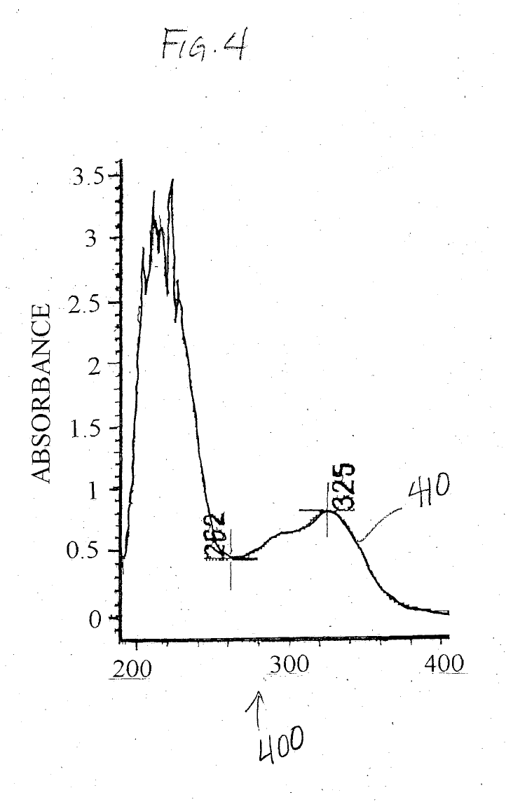

Referring now to FIG. 4, graph 400 shows an absorbance plot for a jojoba extract formed using 100% ethanol extraction of jojoba meal. As curve 410 shows, the 100% ethanol extract of this embodiment of step 150 absorbs ultraviolet radiation at a plurality of wavelengths between 290 nanometers and 380 nanometers with a maximum absorption in that range at about 325 nanometers.

In certain embodiments, step 150 includes extracting the one or more jojoba plant parts with one or more acids. In certain embodiments, step 150 includes extracting the one or more jojoba plant part with one or more acids in combination with one or more alcohols and/or one or more diols and/or one or more polyols. In certain embodiments, the one or more acids of step 150 include, for example, formic acid, acetic acid, propionic acid, and the like. In certain embodiments, the one or more acids of step 150 comprise one or more hydroxy-acids. By “hydroxy-acid,” Applicant means a compound having a carboxylic acid functionality and a hydroxy functionality. In certain embodiments, the one or more hydroxy-acids of step 150 include one or more alpha-hydroxy acids. Such alpha-hydroxy acids include, without limitation, glycolic acid, lactic acid, mandelic acid, malic acid, citric acid, tartaric acid, and combinations thereof. In certain embodiments, the one or more acids of step 150 include one or more beta-hydroxy acids, such as and without limitation, salicylic acid, beta hydroxybutanoic acid, tropic acid, trethocanic acid, and the like, and mixtures thereof. In certain embodiments, the one or more acids of step 150 include one or more alpha hydroxy acids in combination with one or more beta-hydroxy acids.

In certain embodiments, step 150 includes maintaining the pH of the extract at about 3 by small incremental additions of acid. In certain embodiments, step 150 includes maintaining the pH of the extract at about 4 by small incremental additions of acid. In certain embodiments, step 150 includes maintaining the pH of the extract at about 4.5 by small incremental additions of acid.

In certain embodiments the one or more alcohols include, for example, butanol, pentanol, hexanol, and the like. In certain embodiments, the one or more diols include, for example, propylene glycol, polyethylene oxide diol, polypropylene oxide diol, and the like. In certain embodiments, the one or more polyols include, for example, glycerin, carbohydrate acetates, and the like.

Applicant has found that extraction of jojoba plant parts with one or more acids in combination with one or more alcohols/diols/polyols extracts, inter alia, Simmondsin and/or Simmondsin derivatives. A substantial amount of the plurality of jojoba proteins, however, remain in the jojoba plant part, i.e. in the solid materials rather than being removed in the extract.

By “Simmondsin,” Applicant means Compound I wherein R2 is hydrogen, R3 is OH, R4 is OCH₃ and R5 is OCH₃. By “Simmondsin derivative,” Applicant means Compound I wherein R2 is other than hydrogen, R3 is other than OH, R4 is other than OCH₃, and R5 is other than OCH₃.

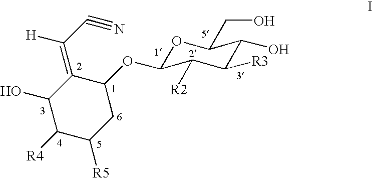

In certain embodiments, the R2 moiety of Compound I comprises a Ferulic acid moiety comprising compound II.

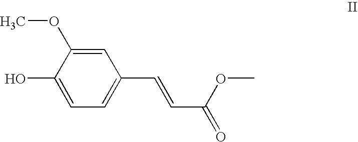

TABLE I summarizes Simmondsin and/or Simmondsin derivatives that comprise various embodiments of Applicant's composition.

TABLE I

| Compound | R2 | R3 | R4 | R5 | Name |
|---|---|---|---|---|---|
| III | H | OH | OCH₃ | OCH₃ | Simmondsin |
| IV | H | OH | OH | OCH₃ | 4-Demethylsimmondsin |
| V | H | OH | OCH₃ | H | 5-Demethylsimmondsin |
| VI | H | OH | OH | OH | Didemethylsimmondsin |
| VII | Compound II | OH | OCH₃ | OCH₃ | Simmondsin 2′-trans-ferulate |
| VIII | OH | Compound II | OCH₃ | OCH₃ | Simmondsin 3′-trans-ferulate |
| IX | Compound II | OH | OH | OCH₃ | 4-Demethylsimmondsin 2′- |
| | | | | | trans-ferulate |
| X | Compound II | OH | OCH₃ | OH | 5-Demethylsimmondsin 2′- |
| | | | | | trans-ferulate |
| XI | Compound II | OH | OH | OH | Didemethylsimmondsin trans- |
| | | | | | ferulate |

It is known that ferulic acid can scavenge free radicals, i.e. function as an antioxidant. See; Yagi K, Ohishi N, Action of ferulic acid and its derivatives as anti-oxidants, Journal of Nutritional Science & Vitaminology, 25(2):127-30, 1979. Embodiments of Applicants' jojoba 5 extract comprising one or more ferulic acid moieties comprise one or more antioxidant compounds. In certain embodiments, Applicants' jojoba extract comprises one or more ferulic-acid-derived moieties in an amount up to about 50 weight percent.

In certain embodiments, the extraction of step 150 is performed multiple times. In certain embodiments, the jojoba plant parts are extracted once. In certain embodiments, the jojoba plant parts are extracted twice. In certain embodiments, the jojoba plant parts are extracted three times. In certain embodiments, the jojoba plant parts are extracted more than three times. In the multiple extraction embodiments, the solid material separated in step 150 is again extracted. In these embodiments, the extracts formed in step 150 are combined.

In certain embodiments, Applicants' method includes step 160. In step 160, the one or more polar solvents used in step 150 are removed from the extract to give up to a 100% solid extract. In these embodiments, the solid extract of step 160 is used in step 170 to formulate Applicants' sunscreen composition.

In embodiments wherein step 150 includes extracting the one or more jojoba plant parts with water, ethanol, or a combination of water and ethanol, then the topical composition of step 170 includes a jojoba extract that absorbs ultraviolet radiation at a plurality of wavelengths between 290 nanometers and 400 nanometers, and that further includes one or more antioxidant compounds in a sufficient amount to reduce reactive oxygen species in the skin when applied topically.

In embodiments wherein step 150 includes extracting the one or more jojoba plant parts with water and/or ethanol in combination with one or more hydroxy acids, then the topical composition of step 170 includes a jojoba extract that absorbs ultraviolet radiation at a plurality of wavelengths between 290 nanometers and 400 nanometers, and that further includes one or more antioxidant compounds in a sufficient amount to reduce reactive oxygen species in the skin when applied topically, in combination with one or more hydroxy acids. Topical application of one or more alpha-hydroxy acids, and/or one or more beta-hydroxy acids, promotes dissolution of adhesions between cells in the upper layers of the skin. Such topical application of one or more hydroxy acids results in shedding dry scales from the skin, i.e. exfoliation. Such exfoliation stimulates the growth of new skin thereby providing a rejuvenated, fresher complexion.

In certain embodiments, Applicant's topical composition further includes one or more non-jojoba-derived skin protectants that reduce skin damage caused by ultraviolet light. These one or more non-jojoba-derived agents include the UVA-type (typical UVA-type sunscreening agents include certain benzophenones and dibenzoyl methanes), the UVB type (typical UVB type sunscreens agents include substituted para-aminobenzoates, alkyl esters of para-methoxycinnamate and certain esters of salicylic acid), or a combination of the two. Generally, the sunscreening agents are used in amounts effective to provide the desired level of protection against UVA and/or UVB radiation. For example, the sunscreening agents are generally used in the amounts of about 2% to about 20% by weight of the total composition. Physical sunscreening agents may also be added to the composition according to the invention. For example, red petrolatum in amounts of about 30% to about 99% by weight of the total composition, or titanium dioxide in amounts of about 2% to about 25% by weight of the total composition can be used. Talc, kaolin, chalk, and precipitated silica can also be used in effective amounts, e.g., about 1% to about 10% by weight of the total composition.

Table I recites the non ojoba-derived sunscreen agents of the present invention and the maximum weight percent of those agents in the sunscreen composition of step 170.

TABLE I

| COMPONENT | WT % |
|---|---|
| Benzophenone-3 (Oxybenzone) | up to 6 |
| Benzophenone-4 (Sulisobenzone) | up to 10 |
| Benzophenone-8 (Dioxybenzone) | up to 3 |
| Butyl Methoxydibenzoylmethane (Avobenzone) | up to 3 |
| Cinoxate | up to 3 |
| Ethylhexyl Dimethyl PABA (Padimate O) | up to 8 |
| Ethylhexyl Methoxycinnamate (Octinoxate) | up to 7.5 |
| Homosalate | up to 15 |
| Menthyl Anthranilate (Meradimate) | up to 5 |
| Octocrylene | up to 10 |
| PABA (Aminobenzoic acid) | up to 15 |
| Phenylbenzimidazole Sulfonic Acid (Ensulizole) | up to 4 |
| TEA-Salicylate (Trolamine salicylate) | up to 12 |
| Titanium Dioxide | up to 25 |
| Zinc Oxide | up to 25 |

In addition to the UV-blocking additives described above, Applicants' formulation of step 170 can include other compounds, including any of the following: (i) emollients, (ii) emulsifiers, (iii) surfactants, (iv) waxes, (v) thickeners, (vi) film formers, (vii) preservatives, and (viii) perfumes.

Emollients may be used according to the invention in amounts which are effective to prevent or relieve dryness. Useful emollients include, without limitation: hydrocarbon oils and waxes; silicone oils; triglyceride esters; acetoglyceride esters; ethoxylated glyceride; alkyl esters; alkenyl esters; fatty acids; fatty alcohols; fatty alcohol ethers; etheresters; lanolin and derivatives; polyhydric alcohols (polyols) and polyether derivatives; polyhydric alcohol (polyol) esters; wax esters; beeswax derivatives; vegetable waxes; phospholipids; sterols; and amides.

Thus, for example, typical emollients include mineral oil, especially mineral oils having a viscosity in the range of 50 to 500 SUS, lanolin oil, mink oil, coconut oil, cocoa butter, olive oil, almond oil, macadamia nut oil, aloa extract, safflower oil, corn oil, liquid lanolin, cottonseed oil, peanut oil, purcellin oil, perhydrosqualene (squalene), castor oil, polybutene, odorless mineral spirits, sweet almond oil, avocado oil, calophyllum oil, ricin oil, vitamin E acetate, mineral spirits, cetearyl alcohol (mixture of fatty alcohols consisting predominantly of cetyl and stearyl alcohols), linolenic alcohol, oleyl alcohol, octyl dodecanol, the oil of cereal germs such as the oil of wheat germ cetearyl octanoate (ester of cetearyl alcohol and 2-ethylhexanoic acid), cetyl palmitate, diisopropyl adipate, isopropyl palmitate, octyl palmitate, isopropyl myristate, butyl myristate, glyceryl stearate, hexadecyl stearate, isocetyl stearate, octyl stearate, octylhydroxy stearate, propylene glycol stearate, butyl stearate, decyl oleate, glyceryl oleate, acetyl glycerides, the octanoates and benzoates of (C12-C15) alcohols, the octanoates and decanoates of alcohols and polyalcohols such as those of glycol and glycerol, and ricin-oleates of alcohols and poly alcohols such as those of isopropyl adipate, hexyl laurate, octyl dodecanoate, dimethicone copolyol, dimethiconol, lanolin, lanolin alcohol, lanolin wax, hydrogenated lanolin, hydroxylated lanolin, acetylated lanolin, petrolatum, isopropyl lanolate, cetyl myristate, glyceryl myristate, myristyl myristate, myristyl lactate, cetyl alcohol, isostearyl alcohol stearyl alcohol, and isocetyl lanolate, and the like.

In certain embodiments, jojoba oil is added as an emollient to Applicants' topical composition in step 170. As discussed above, the jojoba extract of step 150 comprises less than about 1 weight percent jojoba oil. In embodiments of Applicants' method which include steps 110 and 140, the jojoba extract of step 150 is substantially free of jojoba oil.

The composition of the present invention may be provided in an aqueous or non-aqueous solution, suspension or an emulsion (water-in-oil or oil-in-water). The composition may be a skin toner composition, a moisturizing lotion, a sunscreen compositions a skin cleanser, or any other skin treatment composition. The composition may also be used in methods of protecting skin against the harmful effects of ultraviolet radiation, by applying topically to the skin. The composition may be applied before or after exposure to the sun, but is preferably applied prior to sun exposure, for example immediately before sun exposure.

Emulsifiers (i.e., emulsifying agents) are also used in certain aspects of the invention in amounts effective to provide uniform blending of ingredients of the composition. Useful emulsifiers include (i) anionics such as fatty acid soaps, e.g., potassium stearate, sodium stearate, ammonium stearate, and triethanolamine stearate; polyol fatty acid monoesters containing fatty acid soaps, e.g., glycerol monostearate containing either potassium or sodium salt; sulfuric esters (sodium salts), e.g., sodium lauryl 5 sulfate, and sodium cetyl sulfate; and polyol fatty add monoesters containing sulfuric esters, e.g., glyceryl monostearate containing sodium lauryl sulfate; (ii) cationics chloride such as N(stearoyl colamino formylmethyl) pyridium; N-soya-N-ethyl morpholinium ethosulfate; alkyl dimethyl benzyl ammonium chloride; diisobutylphenoxytheoxyethyl dimethyl benzyl ammonium chloride; and cetyl pyridium chloride; and (iii) nonionics such as polyoxyethylene fatty alcohol ethers, e.g., monostearate; polyoxyethylene lauryl alcohol; polyoxypropylene fatty alcohol ethers, e.g., propoxylated oleyl alcohol; polyoxyethylene fatty add esters, e.g., polyoxyethylene stearate; polyoxyethylene sorbitan fatty add esters, e.g., polyoxyethylene sorbitan monostearate; sorbitan fatty add esters, e.g., sorbitan; polyoxyethylene glycol fatty add esters, e.g., polyoxyethylene glycol monostearate; and polyol fatty acid esters, e.g., glyceryl monostearate and propylene glycol monostearate; and: ethoxylated lanolin derivatives, e.g., ethoxylated lanolins, ethoxylated lanolin alcohols and ethoxylated cholesterol.

Surfactants are also used in certain compositions of the invention. Suitable surfactants may include, for example, those surfactants generally grouped as cleansing agents, emulsifying agents, foam boosters, hydrotropes, solubilizing agents, suspending agents and nonsurfactants (facilitates the dispersion of solids in liquids). The surfactants are usually classified as amphoteric, anionic, cationic and nonionic surfactants. Amphoteric surfactants include acylamino adds and derivatives and N-alkylamino adds. Anionic surfactants include: acylamino acids and salts, such as, acylglutamates, acylpeptides, acylsarcosinates, and acyltaurates; carboxylic acids and salts, such as, alkanoic acids, ester carboxylic acids, and ether carboxylic adds; sulfonic adds and salts, such as, acyl isethionates, alkylaryl sulfonates, alkyl sulfonates, and sulfosuccinates; sulfuric add esters, such as, alkyl ether sulfates and allyl sulfates. Cationic surfactants include: alkylamines, alkyl imidazolines, ethoxylated amines, and quaternaries (such as, alkylbenzyldimethylammonium salts, alkyl betaines, heterocylic ammonium salts, and tetra alkylammonium salts). And nonionic surfactants include: alcohols, such as primary alcohols containing 8 to 18 carbon atoms; alkanolamides such as alkanolamine derived amides and ethoxylated amides; amine oxides; esters such as ethoxylated carboxylic acids, ethoxylated glycerides, glycol esters and derivatives, monoglycerides, polyglyceryl, esters, polyhydric alcohol esters and ethers, sorbitan/sorbitol esters, and triesters of phosphoric acid; and ethers such as ethoxylated alcohols, ethoxylated lanolin, ethoxylated polysiloxanes, and propoxylated polyoxyethylene ethers.

Suitable waxes which are useful in accord with the invention include: animal waxes, such as beeswax, spermaceti, or wool wax (lanolin); plant waxes, such as carnauba or candelilla; mineral waxes, such as montan wax or ozokerite; and petroleum waxes, such as paraffin wax and microcrystalline wax (a high molecular weight petroleum wax). Animal, plant, and some mineral waxes are primarily esters of a high molecular weight fatty alcohol with a high molecular weight fatty acid. For example, the hexadecanoic acid ester of tricontanol is commonly reported to be a major component of beeswax. Other suitable waxes according to the invention include the synthetic waxes including polyethylene polyoxyethylene and hydrocarbon waxes derived from carbon monoxide and hydrogen.

Representative waxes also include: ceresin; cetyl esters; hydrogenated jojoba oil; hydrogenated jojoba wax; hydrogenated rice bran wax; Japan wax; jojoba butter; jojoba oil; jojoba wax; munk wax; montan add wax; ouricury wax; rice bran wax; shellac wax; sufurized jojoba oil; synthetic beeswax; synthetic jojoba oils; trihydroxystearin; cetyl alcohol; stearyl alcohol; cocoa butter; fatty acids of lanolin; mono-, di- and triglycerides which are solid at 25° C., e.g., glyceyl tribehenate (a triester of behenic acid and glycerine) and C18-C36 acid triglyceride (a mixture of triesters of C18-C36 carboxylic acids and glycerine) available from Croda, Inc., New York, N.Y. under the tradenames Syncrowax HRC and Syncrowax HGL-C, respectively; fatty esters which are solid at 25° C.; silicone waxes such as methyloctadecaneoxypolysiloxane and poly(dimethylsiloxy)stearoxysiloxane; stearyl mono- and diethanolamide, rosin and its derivatives such as the abietates of glycol and glycerol; hydrogenated oils solid at 25.degree. C.; and sucroglycerides, Thickeners (viscosity control agents) which may be used in effective amounts in aqueous systems include: algin; carbomers such as carbomer 934, 934P, 940 and 941; cellulose gum; cetearyl alcohol, cocamide DEA, dextrin; gelatin; hydroxyethylcellulose; hydroxypropylcellulose; hydroxypropyl methylcellulose; magnesium aluminum silicate; myristyl alcohol; oat flour; oleamide DEA; oleyl alcohol; PEG-7M; PEG14M; PEG-90M; stearamide DEA; Stearamide MEA; stearyl alcohol, tragacanth gum; wheat starch; xanthan gum; and the like in the above list of thickeners, DEA is diethanolamine, and MEA is monoethanolamine. Thickeners (viscosity control agents) which may be used in effective amounts in nonaqueous systems include, aluminum stearates; beeswax; candelilla wax; carnauba; ceresin; cetearyl alcohol; cetyl alcohol; cholesterol; hydrated silica; hydrogenated castor oil; hydrogenated cottonseed oil; hydrogenated soybean oil; hydrogenated tallow glyceride; hydrogenated vegetable oil; hydroxypropyl cellulose; lanolin alcohol; myristyl alcohol; octytdodecyl stearoyl sulfate; oleyl alcohol; ozokerite; microcystalline wax; paraffin; pentaerythrityl tetraoctanoate; polyacrylamide; polybutene; polyethylene; propylene glycol dicaprylate; propylene glycol dipelargonate; stearalkonium hectorite; stearyl alcohol; stearyl stearate; synthetic beeswax; trihydroxystearin; trilinolein; tristearen; zinc stearate; and the like.

Suitable film formers which are used in accord with the invention keep the composition smooth and even and include, without limitation: acrylamide/sodium acrylate copolymer; ammonium acrylates copolymer; Balsam Peru; cellulose gum; ethylene/maleic anhydride copolymer, hydroxyethylcellulose; hydroxypropylcellulose; polyacrylamide; polyethylene; polyvinyl alcohol; pvm/MA copolymer (polyvinyl methylether/maleic anhydride); PVP (polyvinylpyrrolidone); maleic anhydride copolymer such as PA-18 available from Gulf Science and Technology; PVP/hexadecene copolymer such as Ganex V-216 available from GAF Corporation; acryliclacrylate copolymer; and the like.

Generally, film formers can be used in amounts of about 0.1 weight percent to about 10 weight percent of the total composition with about 1 weight percent to about 8 weight percent being preferred and about 0.1 weight percent to about 5 weight percent: being most preferred. Humectants can also be used in effective amounts, including: fructose; glucose; glulamic acid; glycerin; honey; maltitol; methyl gluceth-10; methyl gluceth-20; propylene glycol; sodium lactate; sucrose; and the like.

Preservatives according to certain compositions of the invention include, without limitation: butylparaben; ethylparaben; imidazolidinyl urea; methylparaben; O-phenylphenol; propylparaben; quaternium-14; quaternium-15; sodium dehydroacetate; zinc pyrithione; and the like. The preservatives are used in amounts effective to prevent or retard microbial growth. Generally, the preservatives are used in amounts of about 0.1% to about 1% by weight of the total composition.

Perfumes (fragrance components) and colorants (coloring agents) well known to those skilled in the art may be used in effective amounts to impart the desired fragrance and color to the compositions of the invention.

Other ingredients which can be added or used in amounts effective for their intended use, including: biological additives to enhance performance or consumer appeal such as amino acids, proteins, vanilla, aloe extract, bioflavinoids, and the like; buffering agents, chelating agents such as EDTA; emulsion stabilizers; pH adjusters; opacifying agents; and propellants such as butane carbon dioxide, ethane, hydrochlorofluorocarbons 22 and 142b, hydrofluorocarbon 152a, isobutane, isopentane, nitrogen, nitrous oxide, pentane, propane, and the like.

The ingredients described above—sunscreening agents, emollients, emulsifiers, surfactants, solvents for sunscreening agents, waxes, thickeners, film formers, humectants, preservatives, surfactants, perfumes, coloring agents, biological additives, buffering agents, chelating agents, emulsion stabilizers, opacifying agents, pH adjusters, and propellants—are well known to those skilled in the art. The determination of which ingredients to use to obtain the intended formulations, and the determination of the amounts which may be used to achieve the intended functions and effects of these ingredients are well within the capabilities of those skilled in the art without the need for undue experimentation. Further information may be obtained on these ingredients, for example, by reference to: Cosmetics & Toiletries, Vol. 102, No. 3, March 1987; Balsam, M. S., et al., editors, Cosmetics Science and Technology, 2nd edition, Vol. 1, pp 27-104 and 179-222 Wiley-Interscience, New York, 1972; Cosmetics & Toiletries, Vol. 104, pp 67-111, February 1989; Cosmetics & Toiletries, Vol. 103, No. 12, pp 100-129, December 1988; Nikitakis, J. M., editor, CTFA Cosmetic Ingredient Handbook, First Edition, published by The Cosmetic, Toiletry and Fragrance Association, Inc., Washington, D.C., 1988; Mukhtar, H, editor, Pharmacology of the Skin, CRC Press 1992; and Green; F. J., The Sigma-Aldrich Handbook of Stains, Dyes and Indicators., Aldrich Chemical Company, Milwaukee Wis. 1991.

Examples III and IV are presented to further illustrate to persons skilled in the art how to make and use the invention and to identify certain embodiments thereof. These examples are not intended as limitations, however, upon the scope of the invention, which is defined only by the appended claims.

### EXAMPLE III

Table II recites a formulation for Applicants' hair conditioner comprising a UV-absorbing jojoba extract. In this embodiment, the one or more UV-absorbing, jojoba-derived compounds were extracted from jojoba meal.

TABLE II

| | | | |
|---|---|---|---|
| | | Weight | |
| INGREDIENTS | INCI Name | Percent | Supplier |

PART A

| | | | |
|---|---|---|---|
| Jojoba Oil Colorless | Simmondsia Chenensis | 5.0 | Desert Whale |
| | (Jojoba) Seed Oil | | Jojoba Company |
| Incroquat Behenyl | Cetearyl Alcohol (and) | 2.0 | Croda |
| TMS | Behentrimonium | | |
| | Methosulfate | | |

PART B

| | | | |
|---|---|---|---|
| Deionized Water | Water | 86.5 | |
| Jaguar C-17 | Guar Hydroxypropyl- | 0.2 | Rhodia |
| | Trimonium Chloride | | |
| Citric Acid | Citric Acid | 0.1 | |
| (25% aq. Soln.) | | | |

PART C

| | | | |
|---|---|---|---|
| UV-Absorbing | | 5.0 | Desert Whale |
| Jojoba Extract | | | Jojoba Company |
| Fragrance | Fragrance | 0.2 | |
| Herbalesse AFF | | | |
| #116778 | | | |
| Phenobact | Phenoxyethanol (and) | 1.0 | Alzo |
| | Methylparaben (and) | | |
| | Propylparaben (and) | | |
| | Butylparaben (and) | | |
| | Ethylparaben | | |
| Colorants | As needed | QS | |

The formulation of TABLE II was prepared by adding the Part B water to a suitable vessel. That water was vigorously stirred and the Jaguar C-17 was slowly added. The Jagquar C-17 is used to adjust the viscosity of the formulation. In other embodiments, one or more other Theological additives are used. The aqueous mixture was stirred until uniform. Thereafter, the remainder of the Part B ingredients were added. Thereafter, the mixture was heated to about 70° to 75° C. with stirring.

In a separate vessel, the Part A Ingredients were combined and heated to 75° to 80° C. with mixing. The Part A ingredients were added to the Part B ingredients with stirring. That combined mixture was stirred at 70° to 75° C. for not less than 15 minutes, and until uniform. Thereafter, stirring continued and the mixture was cooled to 35 to 40° C. The Part C ingredients were added to the stirred mixture. Thereafter, the formulation was cooled to the desired fill temperature.

### EXAMPLE IV

Table III recites a formulation for one embodiment of Applicants' sun screen lotion comprising a UV-absorbing jojoba extract. In this embodiment, the one or more UV-absorbing, jojoba-derived compounds were extracted from jojoba meal.

TABLE III

| | | | |
|---|---|---|---|
| | | Weight | |
| INGREDIENTS | INCI Name | Percent | Supplier |

PART A

| | | | |
|---|---|---|---|
| Jojoba Oil Colorless | Simmondsia Chenensis | 5.00 | Desert Whale |
| | (Jojoba) Seed Oil | | Jojoba Company |
| Dermoblock OMC | Octyl | 5.00 | Alzo |
| | Methoxycinnamate | | |
| Crodacol CS-50 | Cetearyl Alcohol | 2.00 | Croda |
| Behenic Acid | Behenic Acid | 0.50 | Croda |

PART B

| | | | |
|---|---|---|---|
| Deionized Water | Water | 78.64 | |
| Glycerin | Glycerin | 2.00 | |
| Pemulen TR-1 | Acrylates/ | 0.30 | Noveon, Inc. |
| | C10-30 Alkyl | | |
| | Acrylate Crosspolymer | | |
| Triethanolamine | Triethanolamine | 0.50 | |
| (99%) | | | |

PART C

| | | | |
|---|---|---|---|
| UV-Absorbing | | 5.00 | Desert Whale |
| Jojoba Extract | | | Jojoba Company |
| Phenobact | Phenoxyethanol (and) | 1.00 | Alzo |
| | Methylparaben (and) | | |
| | Propylparaben (and) | | |
| | Butylparaben (and) | | |
| | Ethylparaben | | |
| Fragrance | Fragrance | 0.06 | |
| AFF #116779 | | | |
| Colorants | As needed | QS | |

The formulation of TABLE III was prepared by combining Part A Ingredients in a suitable vessel and heating to about 75° C. to about 80° C. The water component of Part B was added to a suitable vessel and vigorously agitated while slowly adding the Pemulen TR-1. Stirring was continued until the mixture was uniform. Thereafter, the mixing speed was reduced, and remaining Part B ingredients added. That mixture was heated to about 70 to about 75° C. with stirring.

The Part A ingredients were added to the Part B ingredients with mixing. Stirring was continued for at least 15 min. and until the combined mixture was uniform. Thereafter, the mixture was cooled to about 35 to 40° C. with stirring. The Part C ingredients were added, and the formulation was stirred until uniform. Thereafter, the formulation was cooled to the desired filling temperature with stirring.

The UV-absorbing Jojoba Extract of Examples III and IV was prepared using 200 grams of expeller pressed jojoba meal, 200 milliliters of anhydrous ethanol, and 800 milliliters of deionized water. The 200 grams of jojoba meal was sieved through a ˜60 mesh screen into a 1500 ml beaker. A liter of the above-described alcohol/water mixture was added. The resulting slurry was stirred for about 2.5 hours at room temperature (˜70° F.) using an electric laboratory mixer equipped with a propeller blade (2.5 in diameter) at a speed of about 200 rpm.

The stirring was stopped, and the mixture was covered and held without stirring for about 9.5 hours. Thereafter, the supernatant was decanted and gravity filtered through a paper filter (Ahlstrom 615 fast). The residual solids and remaining liquid was put into a 5 inch Buchner Funnel fitted with a paper filter (Ahlstrom 615). The filter assembly was fitted into a side arm Erlenmeyer vacuum flask, dammed with plastic wrap, and attached for about 0.5 hours to a vacuum source at about 3 mm vacuum. About 800 milliliters of the decanted supernatant and about 100 milliliters of filtrate were combined to form the UV-absorbing Jojoba Extract.

While the preferred embodiments of the present invention have been illustrated in detail, it should be apparent that modifications and adaptations to those embodiments may occur to one skilled in the art without departing from the scope of the present invention as set forth in the following claims.

We claim:

1. A topical composition for reducing skin damage induced by ultraviolet radiation comprising a jojoba extract, wherein said jojoba extract absorbs ultraviolet radiation at a plurality of wavelengths between about 290 nanometers and about 400 nanometers.

2. The topical composition of claim 1, further comprising at least one other skin protectant that reduces the skin damage caused by ultraviolet light.

3. The topical composition of claim 2, further comprising aminobenzoic acid.

4. The topical composition of claim 2, further comprising octyl methoxycinnamate.

5. The topical composition of claim 2, further comprising titanium dioxide.

6. The composition of claim 1, in an aqueous or non-aqueous solution, suspension, a water-in-oil or oil-in-water emulsion.

7. The composition of claim 1, in a skin toner composition, a moisturizing lotion, a sun screen composition, a skin cleanser, a hair conditioner, or other skin treatment composition.

8. The topical composition of claim 1, further comprising an antioxidant comprising a ferulic acid moiety in a sufficient amount to reduce reactive oxygen species in the skin when applied topically.

9. The topical composition of claim 1, wherein said jojoba extract is formed by extracting jojoba plant parts with ethanol.

10. The topical composition of claim 9, wherein said jojoba plant parts comprise jojoba meal.

11. The topical composition of claim 10, wherein said jojoba extract is formed by extracting jojoba meal with a mixture of ethanol and water.

12. The topical composition of claim 11, wherein said jojoba extract is formed by extracting milled jojoba meal with ethanol.

13. A method to prepare a topical composition for reducing skin damage induced by ultraviolet radiation, comprising the steps of:
providing jojoba plant parts;
removing up to about 90 weight percent of the jojoba oil disposed in said jojoba plant parts;
milling said jojoba plant parts;
forming a jojoba extract by extracting said milled jojoba plant parts with one or more polar solvents, wherein said jojoba extract absorbs ultraviolet radiation at a plurality of wavelengths between about 290 nanometers and about 400 nanometers; and
adding at least one other skin protectant that reduces the skin damage caused by ultraviolet light.

14. The method of claim 13, wherein said one or more polar solvents comprise ethanol.

15. The method of claim 13, wherein said one or more polar solvents comprise water.

16. The method of claim 15, wherein said jojoba extract comprises a ferulic acid moiety.
